(19) United States
(12) Patent Application Publication
(10) Pub. No.: US 2023/0114600 A1
(43) Pub. Date: Apr. 13, 2023

(54) TELEHEALTH PROVIDER MANAGEMENT SYSTEM AND METHOD

(71) Applicant: MDLIVE, Inc., Miramar, FL (US)

(72) Inventor: Nakort E. Valles Leon, Weston, FL (US)

(21) Appl. No.: 17/486,251

(22) Filed: Sep. 27, 2021

(51) Int. Cl. G16H 80/00; G16H 40/20

(52) U.S. Cl. CPC G16H 80/00; G16H 40/20

(57) ABSTRACT

A system and method for managing telehealth encounters between providers and patients in a telehealth system are provided. A first group of the providers is provided with an ability to select a first patient of the patients from a virtual waiting room to initiate a first telehealth encounter of the telehealth encounters. An amount of time that the first patient is waiting in the virtual waiting room without being selected for initiation of the first telehealth encounter is tracked. Responsive to the amount of time that the first patient is waiting exceeding a first threshold period of time, a second group of the providers is provided with the ability to select the first patient from the virtual waiting room to initiate the first telehealth encounter.

### BACKGROUND

Many different types of services can be provided via distributed or long-distance systems. For example, telehealth systems can provide health-related services and information via electronic information and telecommunication technologies. These systems can allow for long-distance provider and customer interaction via video conferencing and/or telephone calls. With respect to telehealth systems, healthcare providers may be able to be reached for consultations, medical appointments, etc., for a variety of medical issues.

With respect to telehealth systems, patients may seek an appointment with a healthcare provider via a remote encounter (e.g., a videoconference or teleconference). The patients may be placed into a virtual waiting room until a suitable and available healthcare provider is found. Once the suitable and available healthcare provider is found, the remote encounter may be established, and the patients may remotely interact with the providers to receive medical care, advice, and the like.

### BRIEF DESCRIPTION

In one embodiment, a method for managing telehealth encounters between providers and patients in a telehealth system is provided. The method includes providing a first group of the providers with an ability to select a first patient of the patients from a virtual waiting room to initiate a first telehealth encounter of the telehealth encounters. The method also includes tracking an amount of time that the first patient is waiting in the virtual waiting room without being selected for initiation of the first telehealth encounter, and responsive to the amount of time that the first patient is waiting exceeding a first threshold period of time, providing a second group of the providers with the ability to select the first patient from the virtual waiting room to initiate the first telehealth encounter.

In one embodiment, a telehealth system includes a control system having one or more processors configured to provide a first group of the providers with an ability to select a first patient of the patients from a virtual waiting room to initiate a first telehealth encounter of the telehealth encounters. The control system is configured to track an amount of time that the first patient is waiting in the virtual waiting room without being selected for initiation of the first telehealth encounter and, responsive to the amount of time that the first patient is waiting exceeding a first threshold period of time, the control system is configured to provide a second group of the providers with the ability to select the first patient from the virtual waiting room to initiate the first telehealth encounter.

In one embodiment, a tangible and non-transitory computer readable storage medium is provided. The storage medium has instructions that direct one or more processors to provide a first group of the providers with an ability to select a first patient of the patients from a virtual waiting room to initiate a first telehealth encounter of the telehealth encounters, track an amount of time that the first patient is waiting in the virtual waiting room without being selected for initiation of the first telehealth encounter, and, responsive to the amount of time that the first patient is waiting exceeding a first threshold period of time, provide a second group of the providers with the ability to select the first patient from the virtual waiting room to initiate the first telehealth encounter.

### BRIEF DESCRIPTION OF THE DRAWINGS

FIG. 1 illustrates one example of a telehealth system;

FIG. 2 illustrates one example of changing pools or groups of providers that are available to a patient for initiating a telehealth encounter or session;

FIG. 3 illustrates another example of changing pools or groups of providers that are available to a patient for initiating a telehealth encounter or session;

FIG. 4 illustrates a flowchart of one example of a method for managing providers in a telehealth system; and

FIG. 5 shows a block diagram of a computer system.

In the drawings, reference numbers may be reused to identify similar and/or identical elements.

### DETAILED DESCRIPTION

Some telehealth systems may suffer from inefficiencies in initiating the encounters between the healthcare providers and patients. For example, some telehealth systems may rely on a limited pool of healthcare providers to remotely interact with patients, with the healthcare providers in this limited pool having a contractual arrangement or other arrangement with the entity providing the telehealth system. While there may be other healthcare providers able to engage in the patients via the telehealth system, these other healthcare providers may not have the same arrangement with the entity providing the telehealth system. For example, the healthcare providers in the limited pool may be compensated at a lower amount or rate due to the arrangement with the entity, while the healthcare providers that are outside of the limited pool may be compensated at a higher amount or rate. As a result, the telehealth system may prioritize the healthcare providers in the limited pool over the healthcare providers not within the limited pool or group when establishing remote telehealth services between the healthcare providers and the patients. During high volume times when patient demand for telehealth services is high, the limited pool or group of providers may not be sufficient to provide the telehealth services to the patients. Consequently, some patients may wait exceedingly long times in a virtual waiting room for a remote engagement with a provider. These patients may become discouraged or irritated, leave the virtual waiting room, and potentially not receive the professional medical care or advice that is needed to help the patients in a timely manner.

Example systems and methods for managing healthcare providers in a telehealth system are described herein. The providers described herein may be healthcare providers and the consumers may be patients in one embodiment for ease of illustration and explanation, although not all embodiments of the inventive subject matter are limited to healthcare services. One or more embodiments may be used in connection with other types of remote services, such as legal counseling, financial advisement services, retail sales, or the like. The healthcare providers and patients may interact with each other via a videoconference or teleconference channel (e.g., using electronic communication devices connected over a communication network or channel). Patients may access the telehealth system via an electronic communication device such as a mobile phone, tablet computer, laptop computer, desktop computer, smart television, or the like. The patients can be placed into a virtual waiting room, such as an electronically managed list of patients waiting to interact with a healthcare provider. A telehealth session may be initiated between patients and healthcare providers on a first-come, first-served basis. For each patient, the telehealth system can provide a first group or pool of the providers. If there are more patients waiting in the virtual waiting room than there are available providers, then there may be some patients that are required to continue waiting in the virtual waiting room until telehealth sessions between providers and other patients are completed (and the providers in the first pool or group are free to initiate a telehealth session with the patients that are still waiting).

In one example of the inventive subject matter described herein, for each patient in a virtual waiting room, the telehealth system and method can provide different pools or groups of providers for initiating telehealth sessions with the patient based on how long the patient has been waiting in the virtual waiting room. For example, when a patient initially enters the virtual waiting room, there may be a first pool or group of healthcare providers that separately and remotely interact with other patients already in the waiting room. If the patient remains in the virtual waiting room for longer than a threshold period of time (e.g., three minutes, five minutes, etc., in different examples), then the telehealth system can change the pool or group of healthcare providers for that patient. For example, a second pool or group of healthcare providers may be made available for that patient (and any other patient(s) waiting for longer than the threshold period of time). This second pool or group of healthcare providers can be a larger pool in that there are more providers in the second pool or group than the first pool or group. The providers in the first group may be included in the second group (with additional providers that are not in the first group being included in the second group). Alternatively, the providers in the first and second groups may be different such that no provider in the first group is in the second group. In one embodiment, the telehealth system can continue to track how long the patient is waiting before a remote engagement or session between the patient and a healthcare provider initiates or is completed. As the length of time that the patient waits for the session to begin increases, the pool or group of providers can change (e.g., increase in size). For example, if the patient is waiting for longer than a second threshold period of time, a third pool of providers may become available to the patient; if the patient is waiting for longer than a third threshold period of time, a fourth pool of providers may become available to the patient, and so on. The pools of providers can increase in size or otherwise change as the length of time that the patient is waiting increases. This can reduce how long the patient would otherwise wait (without changing the pool of providers) as more and/or different providers become available over time.

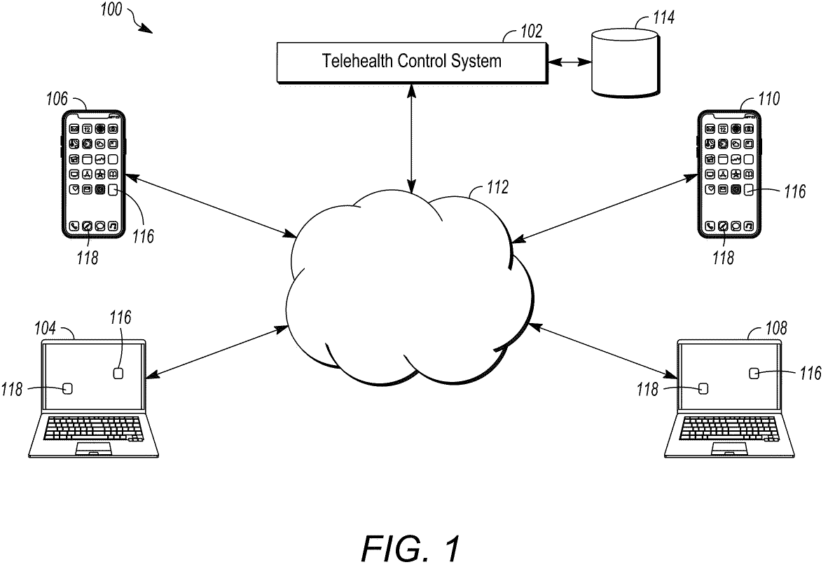

FIG. 1 illustrates one example of a telehealth system 100. The telehealth system 100 may be used to establish and conduct remote encounters between healthcare providers and patients. A remote encounter may include an interaction or session (e.g., meeting, medical appointment, medical consultation, etc.) between a healthcare provider and a patient via video conference or teleconference while the healthcare provider and the patient are in different locations (e.g., different rooms, different buildings, different towns or cities, different zip codes, different states, or different countries). The healthcare provider can be a Doctor of Medicine, Doctor of Osteopathy, a podiatrist, a dentist, a chiropractor, a clinical psychologist, an optometrist, a nurse practitioner, a nurse-midwife, or a clinical social worker. While the examples described herein relate to the telehealth system 100 being used to establish remote encounters between healthcare providers and patients, not all embodiments of the inventive subject matter are limited to telehealth systems. For example, the telehealth system 100 optionally can be used to establish and conduct remote encounters between real estate agents and clients, between attorneys and clients, courts and parties to a legal proceeding, teachers and students, governmental meetings, voter and election systems, or any other service provider and customer who are connected via an electronic communication system.

The telehealth system 100 includes a control system 102 that represents hardware circuitry having and/or connected with one or more processors (e.g., microprocessors, field programmable gate arrays, integrated circuits, etc.) that perform the operations described in connect with the control system 102. The control system 102 communicates (wirelessly and/or via wired connections) with provider computing devices 104, 106 and consumer or patient computing devices 108, 110. The computing devices 104, 108 can represent laptop computers, desktop computers, tablet computers, or the like, and the computing devices 106, 110 can represent mobile phones. While only two patient computing devices and only two provider computing devices are shown, the telehealth system 100 can establish communication between many more patient computing devices and many more provider computing devices.

The control system 102 can manage a virtual encounter between the providers and patients by establishing a communication channel between a computing device 104 or 106 of the provider(s) and a computing device 108 or 110 of the patient. This communication channel can be a videoconference or a teleconference that extends through one or more computer networks 112, such as the Internet, one or more intranets, one or more local area networks, or the like. The computing devices 104, 106, 108, 110 may have software applications 116 installed or otherwise running thereon to establish a secure connection between (a) the provider computing device 104 or 106 and (b) the patient computing device 108 or 110. These software applications 116 can be commercial or proprietary applications 116 used by a company or government to manage the remote encounters between providers and patients. The applications 116 can be installed in internal computer memories of the computing devices 104, 106, 108, 110 or may be accessed via web pages 118. One example of such a software application 116 or service is MDLIVE healthcare services that provides remote healthcare services e.g., via telephone, video, email, mobile devices, or a global computer network. The secure connection can extend through the network(s) 112 to ensure confidentiality of the information communicated between the provider and the patient. For example, the videoconference or teleconference channel can extend through one or more digital subscriber lines, cable modems, network fibers, wireless networks, satellite networks, broadband over powerline connections, etc., using the transmission control protocol over Internet protocol, or another protocol.

A patient can submit a request for a virtual encounter to the control system 102 using the software application 116 installed on or accessible on a website 118 via a patient computing device 108 or 110. In FIG. 1, reference number 118 can represent this website that is accessed by an Internet browser. This request can identify the patient, one or more health consultation needs of the patient, and other information. The health consultation needs may identify health issues or questions that the patient wishes to have resolved, answered, or otherwise addressed by a remote encounter with a provider. The control system 102 can use this information to identify one or more appropriate providers. For example, if the request identifies an issue related to the skin of the patient, then the control system 102 can determine that the appropriate provider is a dermatologist. if the request identifies an issue related to diabetes mellitus, then the control system 102 can determine that the appropriate provider is an endocrinologist, and so on. The control system 102 may only send notifications to providers for the virtual encounters where the providers have education, expertise, training, and/or certification to provide service to treat the identified issue.

The control system 102 can include or have access to a tangible and non-transitory computer readable medium, such as a database within a computer memory 114, that stores different specialties of the providers, contact information (e.g., phone numbers) of the providers, and so on. The control system 102 can refer to information stored in the memory 114 to determine appropriate providers with which the videoconference or teleconference with the patient can be provided (based on information included in the request submitted by the patient). This memory 114 optionally can store associations or connections between the providers and different groups or pools of the providers. For example, the memory 114 can store identifying information of the providers with different groups of the providers. This identifying information can include provider names, identifications of provider computing devices, Internet Protocol addresses of the provider computing devices, other network addresses of the provider computing devices, login/password combinations of the providers, other identifying information of the providers, etc.

Many patients may seek virtual appointments with the same type of physician (e.g., physicians with the same medical specialty) at the same time. The control system 102 may place these patients in a queue, or virtual waiting room. Providers can then send a message to the control system 102 (using the provider computing devices 104, 106) to accept a patient for a virtual encounter, e.g., through a first communication channel or through a secondary communication channel. The control system 102 can then establish a connection between the provider computing device 104 or 106 and the patient computing device 108 or 110 via the network(s) 112, such as a teleconference or videoconference, for the remote encounter to occur. In one example, the encounter or session involving a provider and a patient begins once both the provider and the patient are able to see and hear each other (via cameras, microphones, and speakers of the respective computing devices). In another example, the encounter or session involving a provider and a patient begins once both the provider and the patient are able to hear each other (in the event that one or both of the provider and patient does not have a camera).

The control system 102 can track waiting times of the patients in the virtual waiting room. For example, the control system 102 can begin a timer or record the time when a patient computing device 108, 110 submits the request for the virtual encounter to the control system 102 using the software application 116 or the website 118. The control system 102 can then monitor or track how long each patient is waiting in the virtual waiting room without an encounter or session beginning. The control system 102 can repeatedly compare the amount of time since the patient began waiting in the virtual waiting room (or the time since the request was submitted) to one or more waiting time thresholds. If the time that a patient is waiting in the virtual waiting room exceeds one or more of these thresholds, then the pool or group of providers available to that patient to initiate the encounter or session may change.

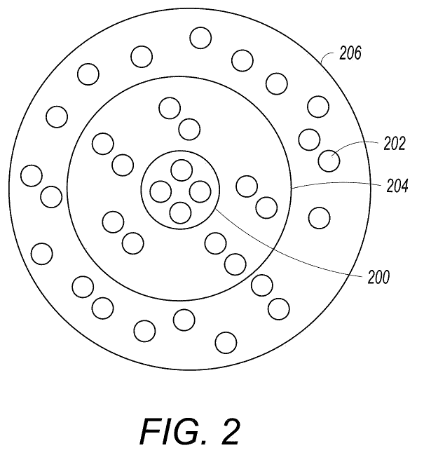

FIG. 2 illustrates one example of changing pools or groups of providers that are available to a patient for initiating a telehealth encounter or session. When a patient enters the virtual waiting room, the telehealth control system 102 (shown in FIG. 1) may only have a first set, group, or pool 200 of providers 202 available to that patient for the telehealth encounter. The providers 202 in other sets, groups, or pools 204, 206 of providers 202 may not be available to that patient yet. For example, the patient may be placed into a waiting list or queue from which the providers 202 in the first pool can select patients to initiate the telehealth encounter. If there are sufficient providers 202 in the first pool 200 and/or patient demand is not too great, then the patient may not need to wait long before being selected by a provider (using his or her computing device) to initiate the telehealth session via the provider and patient computing devices. For example, the patient may wait less than a minute before the telehealth session or encounter begins. But if there are too few providers 202 in the first pool 200 and/or patient demand is large, then the patient may need to wait a longer time before being selected by a provider to initiate the telehealth session via the provider and patient computing devices. For example, the patient may wait more than a minute to be selected for the telehealth session.

If the control system 102 determines that a patient has been waiting in the virtual waiting room for more than a first threshold period of time (e.g., one minute, two minutes, three minutes, or the like, in different embodiments), then the control system 102 can make the providers 202 in a larger, second group 204 of providers 202 available to the patient. For example, prior to the patient waiting for longer than the first threshold period of time, only the providers 202 in the first, smaller group 200 may be allowed by the control system 102 to select the patient to begin the telehealth session or encounter. Once the patient has waited for longer than the first threshold period of time, however, the control system 102 may allow the providers 202 in the larger, second group 204 to select the patient for the telehealth session or encounter. As shown in FIG. 2, the second group 204 may include the providers 202 in the first group 200 and include additional providers 202 that are in the second group 204 but not the first group 200.

The patient may be selected by a provider 202 in the first or second group 200, 204. But the patient alternatively may continue waiting for the telehealth session or encounter to begin if patient demand is high and/or there are too few providers 202 in the first and second groups 200, 204 to meet patient demand. If the patient continues to wait for longer than a second threshold period of time before the telehealth session or encounter begins, the control system 102 can make the providers 202 in a larger, third group 206 of the providers 202 available to the patient. For example, prior to the patient waiting for longer than the second threshold period of time, only the providers 202 in the first and second groups 200, 204 may be allowed by the control system 102 to select the patient to begin the telehealth session or encounter. Once the patient has waited for longer than the second threshold period of time, however, the control system 102 may allow the providers 202 in the larger, third group 206 to select the patient for the telehealth session or encounter. As shown in FIG. 2, the third group 206 may include the providers 202 in the first group 200, the providers 202 in the second group 204, and additional providers 202 that are in the third group 206 but not the first group 200 and not in the second group 204. The number of providers 202 in each group 200, 204, 206 shown in FIG. 2 is provided as one example. One or more of the groups 200, 204, 206 may include a greater number or fewer of the providers 202 than what is shown in FIG. 2.

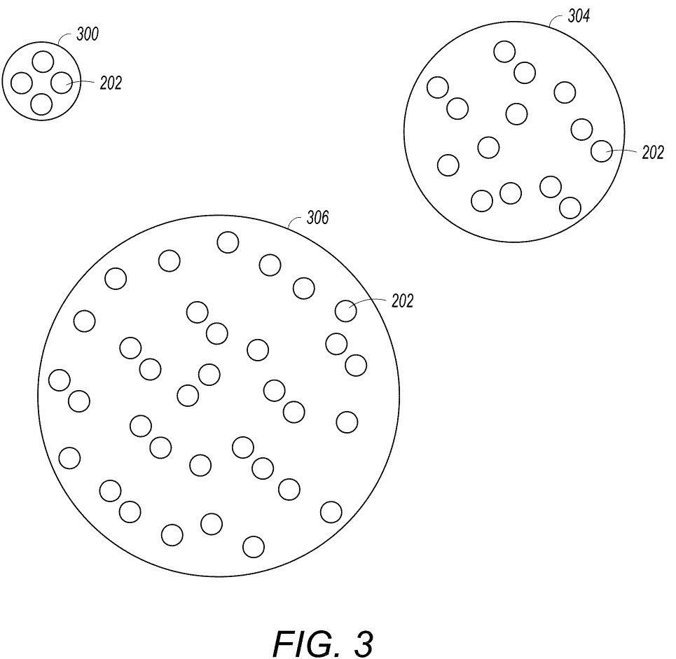

FIG. 3 illustrates another example of changing pools or groups of providers that are available to a patient for initiating a telehealth encounter or session. When a patient enters the virtual waiting room, the telehealth control system 102 may only have a first set, group, or pool 300 of the providers 202 available to that patient for the telehealth encounter. The providers 202 in other sets, groups, or pools 304, 306 of providers 202 may not be available to that patient yet. The patient may be placed into a waiting list or queue from which the providers 202 in the first pool 300 can select patients to initiate the telehealth encounter. If there are sufficient providers 202 in the first pool 300 and/or patient demand is not too great, then the patient may not need to wait long before being selected by a provider to initiate the telehealth session via the provider and patient computing devices. But if the patient waits longer than the first threshold period of time, then the control system 102 can make the providers 202 in a second group 304 of the providers 202 available to the patient, as described above. If the patient continues to wait for longer than a second threshold period of time, then the control system 102 can make the providers 202 in a third group 306 of the providers 202 available to the patient, and so on.

In contrast to the example shown in FIG. 2, the groups 300, 304, 306 of the providers 202 in the example of FIG. 3 do not include each other. That is, no provider 202 in one group 300, 304, or 306 is in any other group 300, 304, 306. The groups 300, 304, 306 in the example of FIG. 3 are non-overlapping or mutually exclusive groups, while the groups 200, 204, 206 in the example of FIG. 2 are overlapping groups. The mutually exclusive groups may be used by the control system 102 to provide the patients with access to different providers 202 as the wait time for the patients increases. For example, based on information provided by the patient via the patient computing device, the control system 102 may provide the patient with access to the group 304 and/or 306 after waiting in the virtual waiting room, with the group 304 and/or 306 including providers 202 having specialties that correspond with the information provided by the patient. If a patient indicates that he or she is suffering from symptoms associated with a respiratory virus or condition, then the control system 102 may initially allow the group 300 of the providers 202 to select the patient from the waiting room. But if the patient waits too long for a telehealth session or encounter, the control system 102 may allow the group 304 of providers 202 to select the patient for the telehealth session or encounter, where the providers 202 in the group 304 specialize in respiratory viruses. This approach can prevent providers 202 with certain medical specialties (e.g., based on work experience and/or education of the providers 202) from being able to select patients until the patients have been waiting too long in the virtual waiting room.

The control system 102 can change the sequence in which the groups 200, 204, 206 or 300, 304, 306 of providers 202 are given the ability to select the patient based on the information provided by the patient. For example, the control system 102 may initially provide the group 300 of general practitioner physicians with the ability to select the patient for the telehealth interaction. After the patient waits longer than the first threshold period of time, the control system 102 may then grant the group 306 of providers 202 with the ability to select the patient (based on specialties of the providers 202) instead of the group 304.

The control system 102 can provide the groups of providers 202 with the ability to select the patient for the telehealth interaction in a different sequence than the ones previously described. Instead of each subsequent group 204, 206 or group 304, 306 having more providers 202 than the prior group 200, 204 or group 300, 304, a subsequent group may have fewer providers than a prior group. For example, the group 306 may initially have the ability to select the patient, followed by the group 300 (after the patient waits the first threshold period of time without being selected), followed by the group 304 (after the patient waits the second threshold period of time without being selected), and so on.

As described above, existing telehealth computing systems suffer from inefficiencies that can cause patients to wait extended periods of time for a telehealth encounter with a provider. The inventive systems and methods described herein improve operation of these telehealth computing systems by more efficiently managing which providers are able to select patients for the telehealth encounters based on how long the patients have been waiting for an interaction.

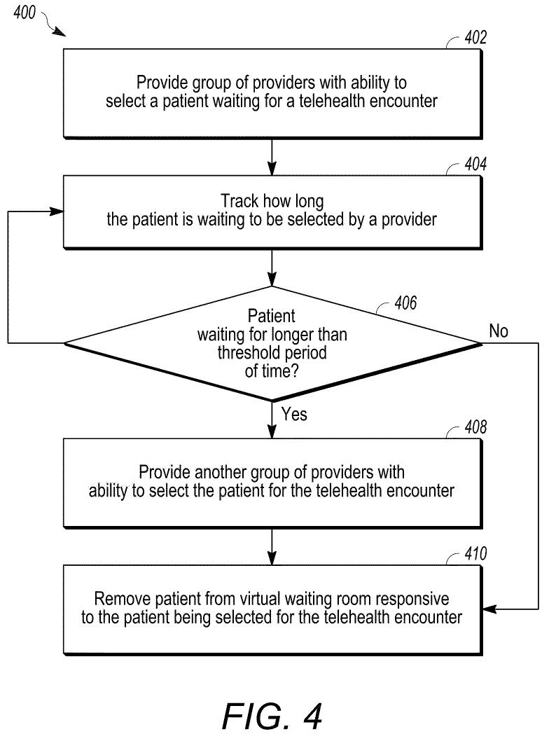

FIG. 4 illustrates a flowchart of one example of a method 400 for managing providers in a telehealth system. The method 900 can represent operations performed by the control system 102 shown in FIG. 1. At 402, a group of providers is given the ability to select a patient from a virtual waiting room for a telehealth encounter or session (e.g., a telehealth medical appointment or meeting with a doctor or other healthcare provider). The control system 102 can prevent other providers 202 (that are outside of this group) from selecting the patient, such as by not including the patient in a list, table, or the like, that is presented to the other providers 202 for selecting the patient, while the patient is included in the list, table, or the like, that is presented to the providers 202 in the group.

At 404, the time that the patient is waiting in the virtual waiting room to be selected for the telehealth encounter is tracked. The control system 102 can include or reference a clock, timer, or the like, to determine how long the patient has been waiting in the virtual waiting room. At 406, if the patient is waiting longer than a threshold period of time, the control system 102 may change which providers 202 are able to select the patient to potentially reduce the amount of additional time that the patient continues to wait for the telehealth encounter (e.g., reduce relative to not changing the group of providers 202 that can select the patient for the encounter). As a result, flow of the method 400 can proceed toward 408. On the other hand, if the patient does not wait for longer than the threshold period of time before being selected, then the patient has been selected by a provider in the group having the ability to select the patient or the patient has otherwise left the virtual waiting room. As a result, flow of the method 400 can proceed toward 410.

At 408, another group of providers is given the ability to select the patient for the telehealth encounter or session. The control system 102 may select a larger group 204, 206, 304, 306 of providers 202 (relative to the prior group having the ability to select the patient), may select a group of providers 202 having specialties that are relevant to the medical issue or information provided by the patient, or the like. Flow of the method 400 can then return toward 404 and repeat one or more times to provide different groups of providers with the ability to select the patient as the wait time for the patient gets longer and longer. At 410, the patient is removed from the virtual waiting room. For example, the patient may no longer be displayed in a list, table, or the like, on the provider computing devices by the control system 102 for selection by providers 202. This can occur once the patient has been selected by a provider or has left the virtual waiting room.

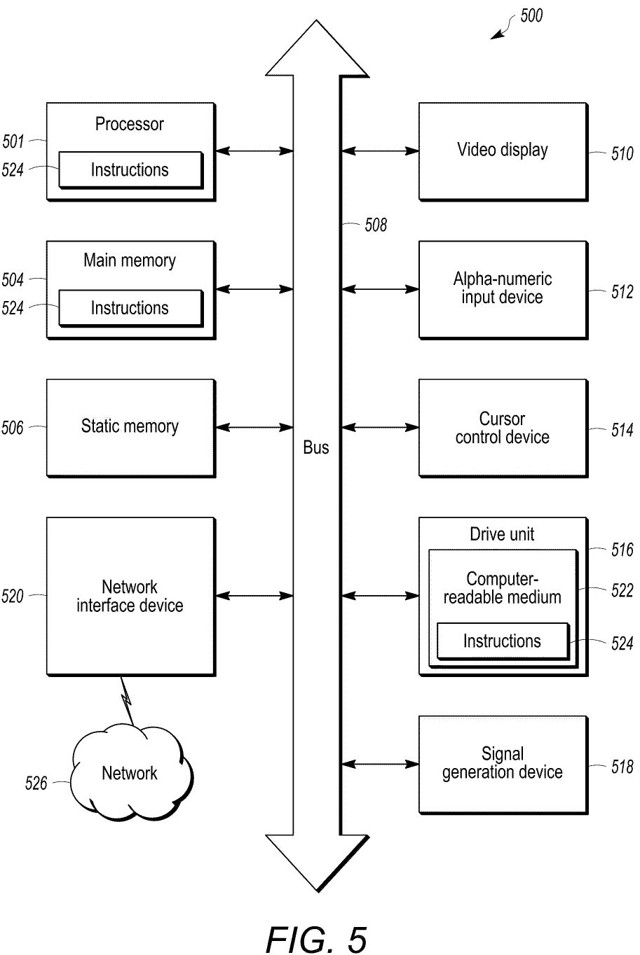

FIG. 5 shows a block diagram of a computer system 500 within which a set of instructions may be executed causing the machine to perform any one or more than one methods, processes, operations, or methodologies discussed herein. For example, the system 500 may represent the control system 102 shown in FIG. 1. The devices 506-530, for example, may include the functionality of the computer system 500. These devices and systems are dedicated to performing any one or more than one methods, processes, operations, or methodologies discussed herein.

In an example embodiment, the machine operates as a standalone device or may be connected (e.g., networked, etc.) to other machines. In a networked deployment, the machine may operate in the capacity of a server or a client machine in server-client network environment, or as a peer machine in a peer-to-peer (or distributed) network environment.

The example computer system 500 includes a processor 502 (e.g., a central processing unit (CPU), a graphics processing unit (GPU) or both, etc.), a main memory 504 and a static memory 506, which communicate with each other via a bus 508. The computer system 500 further includes a video display unit 510 (e.g., a liquid crystal display (LCD) or a cathode ray tube (CRT), etc.). The computer system 500 also includes an alphanumeric input device 512 (e.g., a keyboard, etc.), a cursor control device 514 (e.g., a mouse, etc.), a drive unit 516, a signal generation device 518 (e.g., a speaker, etc.) and a network interface device 520.

The drive unit 516 includes a computer readable medium 522 on which is stored one or more than one sets of instructions 524 (e.g., software, etc.) embodying any one or more than one methodologies or functions described herein. The instructions 524 may also reside, completely or at least partially, within the main memory 504 and/or within the processor 502 during execution thereof by the computer system 500, the main memory 504 and the processor 502 also constituting non-transitory computer readable media. When loaded with the instructions 524, the processor 502 is a machine dedicated to only the present processes and methodologies.

The instructions 524 may further be transmitted or received over a network 526 via the network interface device 520. The instructions can implement multiple communication channels to a provider, e.g., to multiple provider devices and the telehealth control system with one channel being a patient channel that provides data related to the patient and providing connection directly to the patient and a second channel that is a packet channel to provide text messages (e.g., non-video or non-audio communication). The network 526 can represent the network(s) 112 shown in FIG. 1. While the computer-readable medium 522 is shown in an example embodiment to be a single medium, the term “computer-readable medium” should be taken to include a single medium or multiple media (e.g., a centralized or distributed database, and/or associated caches and servers, etc.) that store the one or more than one sets of instructions. The term “computer-readable medium” shall also be taken to include any medium that is capable of storing or encoding a set of instructions for execution by the machine and that cause the machine to perform any one or more than one methodologies of the present disclosure. The term “computer-readable medium” shall accordingly be taken to include, but not be limited to, solid-state memories, and optical media, and magnetic media. In some embodiments, the computer-readable medium is a non-transitory computer-readable medium. In other examples, a computer-readable medium is any medium that satisfies statutory requirements and stores instructions for use by a machine.

The present disclosure uses the term message for describing communication over a secondary communication channel to and from the provider devices. In an example embodiment, the message is not an audio signal. In an example embodiment, the message is not a video signal. Messages can be SMS text messages in an example embodiment. The messages can be packets including a header (including sender device and recipient device identification data) and a body of data. The use of a packet for communication on the second channel can allow for the packet to require less bandwidth to route the information to and from the provider device. Routing the packets can use internet protocol (e.g., IPv4, IPv6 and the like) to transmit the packets. The packet replies from the provider device can be a selection reply, e.g., a single character, that in and of itself does not provide any protected or sensitive information and can be interpreted by the telehealth system.

The use of the first communication channel and the second communication channel can provide for more efficient connection between provider devices and patient devices, as well as computers and communication channels. The first channel can be a private, encrypted channel, which may include protected, private data, which can be through the telehealth system connecting the patient device and the selected provider device. The first channel can provide for menus or selectable information. The second channel is a communication between the telehealth system and provider device, which may not be the same device on which a provider can provide the telehealth consultation with the patient. The second channel can be point to point communication channel over a public communication system and may include data that does not include any protected, private data. The initial message over the second channel can provide instructions for how to reply to indicate a specific action that is to occur over the first channel. The reply message can include the indication of the specific action, e.g., using a single digit or single character. The indication can

In the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, present disclosure may lie in less than all features of a single disclosed embodiment. Thus, the following claims are hereby incorporated into the Detailed Description, with each claim standing on its own as a separate embodiment.

The foregoing description is merely illustrative in nature and is in no way intended to limit the disclosure, its application, or uses. The broad teachings of the disclosure can be implemented in a variety of forms. Therefore, while this disclosure includes particular examples, the true scope of the disclosure should not be so limited since other modifications will become apparent upon a study of the drawings, the specification, and the following claims. It should be understood that one or more steps within a method may be executed in different order (or concurrently) without altering the principles of the present disclosure. Further, although each of the embodiments is described above as having certain features, any one or more of those features described with respect to any embodiment of the disclosure can be implemented in and/or combined with features of any of the other embodiments, even if that combination is not explicitly described. In other words, the described embodiments are not mutually exclusive, and permutations of one or more embodiments with one another remain within the scope of this disclosure.

Spatial and functional relationships between elements (for example, between modules) are described using various terms, including “connected,” “engaged,” “interfaced,” and “coupled.” Unless explicitly described as being “direct,” when a relationship between first and second elements is described in the above disclosure, that relationship encompasses a direct relationship where no other intervening elements are present between the first and second elements, and also an indirect relationship where one or more intervening elements are present (either spatially or functionally) between the first and second elements. As used herein, the phrase at least one of A, B, and C should be construed to mean a logical (A OR B OR C), using a non-exclusive logical OR, and should not be construed to mean “at least one of A, at least one of B, and at least one of C.”

In the figures, the direction of an arrow, as indicated by the arrowhead, generally demonstrates the flow of information (such as data or instructions) that is of interest to the illustration. For example, when element A and element B exchange a variety of information but information transmitted from element A to element B is relevant to the illustration, the arrow may point from element A to element B. This unidirectional arrow does not imply that no other information is transmitted from element B to element A. Further, for information sent from element A to element B, element B may send requests for, or receipt acknowledgements of, the information to element A. The term subset does not necessarily require a proper subset. In other words, a first subset of a first set may be coextensive with (equal to) the first set.

In this application, including the definitions below, the term “module” or the term “controller” may be replaced with the term “circuit.” The term “module” may refer to, be part of, or include processor hardware (shared, dedicated, or group) that executes code and memory hardware (shared, dedicated, or group) that stores code executed by the processor hardware.

The module may include one or more interface circuits. In some examples, the interface circuit(s) may implement wired or wireless interfaces that connect to a local area network (LAN) or a wireless personal area network (WPAN). Examples of a LAN are Institute of Electrical and Electronics Engineers (IEEE) Standard 802.11-2016 (also known as the WIFI wireless networking standard) and IEEE Standard 802.3-2015 (also known as the ETHERNET wired networking standard). Examples of a WPAN are the BLUETOOTH wireless networking standard from the Bluetooth Special Interest Group and IEEE Standard 802.15.4.

The module may communicate with other modules using the interface circuit(s). Although the module may be depicted in the present disclosure as logically communicating directly with other modules, in various implementations the module may actually communicate via a communications system. The communications system includes physical and/or virtual networking equipment such as hubs, switches, routers, and gateways. In some implementations, the communications system connects to or traverses a wide area network (WAN) such as the Internet. For example, the communications system may include multiple LANs connected to each other over the Internet or point-to-point leased lines using technologies including Multiprotocol Label Switching (MPLS) and virtual private networks (VPNs).

In various implementations, the functionality of the module may be distributed among multiple modules that are connected via the communications system. For example, multiple modules may implement the same functionality distributed by a load balancing system. In a further example, the functionality of the module may be split between a server (also known as remote, or cloud) module and a client (or, user) module.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of a non-transitory computer-readable medium are nonvolatile memory devices (such as a flash memory device, an erasable programmable read-only memory device, or a mask read-only memory device), volatile memory devices (such as a static random access memory device or a dynamic random access memory device), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

The computer programs include processor-executable instructions that are stored on at least one non-transitory computer-readable medium. The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc.

The computer programs may include: (i) descriptive text to be parsed, such as HTML (hypertext markup language), XML (extensible markup language), or JSON (JavaScript Object Notation), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective C, Swift, Haskell, Go, SQL, R, Lisp, Java®, Fortran, Perl, Pascal, Curl, OCaml, Javascript®, HTML5 (Hypertext Markup Language 5th revision), Ada, ASP (Active Server Pages), PHP (PHP: Hypertext Preprocessor), Scala, Eiffel, Smalltalk, Erlang, Ruby, Flash®, Visual Basic®, Lua, MATLAB, SIMULINK, and Python®.

None of the elements recited in the claims is intended to be a means-plus-function element within the meaning of 35 U.S.C. §112(f) unless an element is expressly recited using the phrase “means for,” or in the case of a method claim using the phrases “operation for” or “step for.”

The methods described herein do not have to be executed in the order described, or in any particular order. Moreover, various activities described with respect to the methods identified herein can be executed in serial or parallel fashion. Although “End” blocks may be shown in the flowcharts, the methods may be performed continuously.

In the foregoing Detailed Description, it can be seen that various features are grouped together in a single embodiment for the purpose of streamlining the disclosure. This method of disclosure is not to be interpreted as reflecting an intention that the claimed embodiments require more features than are expressly recited in each claim. Rather, as the following claims reflect, present disclosure may lie in less than all features of a single disclosed embodiment. Thus, the following claims are hereby incorporated into the Detailed Description, with each claim standing on its own as a separate embodiment.

What is claimed is:

1. A method for managing telehealth encounters between providers and patients in a telehealth system, the method comprising:
providing a first group of the providers with an ability to select a first patient of the patients from a virtual waiting room to initiate a first telehealth encounter of the telehealth encounters;
tracking an amount of time that the first patient is waiting in the virtual waiting room without being selected for initiation of the first telehealth encounter; and
responsive to the amount of time that the first patient is waiting exceeding a first threshold period of time, providing a second group of the providers with the ability to select the first patient from the virtual waiting room to initiate the first telehealth encounter.

2. The method of claim 1, further comprising:
continuing tracking of the amount of time that the first patient is waiting in the virtual waiting room without being selected for initiating of the first telehealth encounter; and
responsive to the amount of time that the first patient is waiting exceeding a second threshold period of time, providing a third group of the providers with the ability to select the first patient from the virtual waiting room to initiate the first telehealth encounter.

3. The method of claim 1, wherein the first group of the providers is included in the second group of the providers.

4. The method of claim 1, wherein the first group of the providers and the second group of the providers are mutually exclusive groups.

5. The method of claim 1, wherein the second group of the providers includes more of the providers than the first group.

6. The method of claim 1, wherein the second group of the providers includes fewer of the providers than the first group.

7. The method of claim 1, wherein the first group of the providers includes general practitioner providers and the second group of the providers includes the providers with specialties.

8. A telehealth system comprising:
a control system having one or more processors configured to provide a first group of the providers with an ability to select a first patient of the patients from a virtual waiting room to initiate a first telehealth encounter of the telehealth encounters,
the control system configured to track an amount of time that the first patient is waiting in the virtual waiting room without being selected for initiation of the first telehealth encounter and, responsive to the amount of time that the first patient is waiting exceeding a first threshold period of time, the control system is configured to provide a second group of the providers with the ability to select the first patient from the virtual waiting room to initiate the first telehealth encounter.

9. The telehealth system of claim 8, wherein the control system is configured to continue tracking the amount of time that the first patient is waiting in the virtual waiting room without being selected for initiating of the first telehealth encounter, and responsive to the amount of time that the first patient is waiting exceeding a second threshold period of time, the control system is configured to provide a third group of the providers with the ability to select the first patient from the virtual waiting room to initiate the first telehealth encounter.

10. The telehealth system of claim 8, wherein the first group of the providers is included in the second group of the providers.

11. The telehealth system of claim 8, wherein the first group of the providers and the second group of the providers are mutually exclusive groups.

12. The telehealth system of claim 8, wherein the second group of the providers includes more of the providers than the first group.

13. The telehealth system of claim 8, wherein the second group of the providers includes fewer of the providers than the first group.

14. The telehealth system of claim 8, wherein the first group of the providers includes general practitioner providers and the second group of the providers includes the providers with specialties.

15. A tangible and non-transitory computer readable storage medium having instructions that direct one or more processors to:
provide a first group of the providers with an ability to select a first patient of the patients from a virtual waiting room to initiate a first telehealth encounter of the telehealth encounters;
track an amount of time that the first patient is waiting in the virtual waiting room without being selected for initiation of the first telehealth encounter; and
responsive to the amount of time that the first patient is waiting exceeding a first threshold period of time, provide a second group of the providers with the ability to select the first patient from the virtual waiting room to initiate the first telehealth encounter.

16. The storage medium of claim 15, wherein the instructions direct the one or more processors to continuing tracking the amount of time that the first patient is waiting in the virtual waiting room without being selected for initiating of the first telehealth encounter and, responsive to the amount of time that the first patient is waiting exceeding a second threshold period of time, provide a third group of the providers with the ability to select the first patient from the virtual waiting room to initiate the first telehealth encounter.

17. The storage medium of claim 15, wherein the first group of the providers is included in the second group of the providers.

18. The storage medium of claim 15, wherein the first group of the providers and the second group of the providers are mutually exclusive groups.

19. The storage medium of claim 15, wherein the second group of the providers includes more of the providers than the first group.

20. The storage medium of claim 15, wherein the second group of the providers includes fewer of the providers than the first group.
